(19) United States
(12) Patent Application Publication
(10) Pub. No.: US 2024/0315706 A1
(43) Pub. Date: Sep. 26, 2024

(54) IMPROVEMENTS IN AND RELATING TO MOUNTING SYSTEMS

(71) Applicant: DEPUY IRELAND UNLIMITED COMPANY, RINGASKIDDY, CO CORK (IE)

(72) Inventors: MICHAEL REEVE, LEEDS (GB); NEIL WOOLLEN, WEST YORKSHIRE (GB)

(21) Appl. No.: 18/259,950

(22) PCT Filed: Feb. 11, 2022
(86) PCT No.: PCT/EP2022/053345

§ 371 Date: Jun. 29, 2023

(30) Foreign Application Priority Data
Feb. 11, 2021 (GB) 2101870.0

(51) Int. Cl. A61B 17/16; A61B 17/00; A61B 90/00; B23B 31/117

(52) U.S. Cl. CPC A61B 17/162; B23B 31/1175; A61B 2017/00473; A61B 2090/0807

(57) ABSTRACT

Mounting systems for connection to a surgical device, kits including those and the surgical devices and methods of use are disclosed. The mounting system comprising; an elongate body having a proximal end and a distal end; an outer sleeve being provided towards the distal end of the elongate body, the outer sleeve defining an internal bore; an inner element with a distal end facing the internal bore, the inner element providing one or more arms that extend from the distal end of the inner element into the bore, the one or more arms being spaced from the outer sleeve to define a gap between a side of one or more of the arms and the outer sleeve, the inner element at least partially defining a second bore; a locking element slidably provided in the second bore, the locking element having a first state where the locking element is provided in a first position adjacent to a distal portion of one or more of the arms and a second state where the locking element is retracted from the first position adjacent to a distal portion of one or more of the arms.

### TECHNICAL FIELD

This disclosure concerns improvements in and relating to mounting systems, particularly for surgical procedural elements, including those using in orthopaedic surgery.

### BACKGROUND

WO2012/040183 provides a loader which holds an end effector, such as a jaw actuator, of a surgical instrument before use and a handle unit on which the end effector can be mounted and then withdrawn from the loader for use in surgery. Axial retention of the end effector and handle unit are provided, with only a limited ability to transmit low levels of torque. The typical end effectors of interest are relatively short and so only limited resistance to flexing is provided.

It is amongst the potential, but non-limiting aims of the present disclosure to provide a mounting system capable of transferring high levels, primarily torque, but also axial loading [compressive and tensile] to a surgical procedural element, such as a reamer. It is amongst the potential, but non-limiting aims of the present disclosure to provide a mounting system which is resistant to flexing of the mounting system and reamer when connected together. It is amongst the potential but non-limiting aims of the present disclosure to provide a connection between the mounting system and the surgical procedural element that has a low cross-sectional profile, for instance no greater than that of the maximum cross-sectional profile of the surgical procedural instrument. It is amongst the potential, but non-limiting aims of the present disclosure to provide for an accurate and/or resilient and/or quick attachment and detachment between a mounting system and a surgical procedural element.

### SUMMARY

According to a first aspect of the disclosure there is provided a mounting system for connection to a surgical procedural element, the mounting system comprising;
i) an elongate body having a proximal end and a distal end;
ii) an outer sleeve being provided towards the distal end of the elongate body, the outer sleeve defining an internal bore;
iii) an inner element with a distal end facing the internal bore, the inner element providing one or more arms that extend from the distal end of the inner element into the bore, the one or more arms being spaced from the outer sleeve to define a gap between a side of one or more of the arms and the outer sleeve, the inner element at least partially defining a second bore;
iv) a locking element slidably provided in the second bore, the locking element having a first state where the locking element is provided in a first position adjacent to a distal portion of one or more of the arms and a second state where the locking element is retracted from the first position adjacent to a distal portion of one or more of the arms.

The outer sleeve may have a distal end and one or more arms may have distal ends. The distal end of one or more or all arms may be recessed within the internal bore. The distal end of the outer sleeve may extend beyond the distal end of one or more arms. The distal end of the outer sleeve may extend beyond the distal ends of one or more arms by at least a quarter of the length of one or more arms, potentially by at least a third of the length of one or more arms.

The outer sleeve may be an annular outer sleeve. The outer sleeve may have a circular external cross-section. The outer sleeve may have a first internal surface portion. The first internal surface portion may have a consistent cross-sectional profile. The first internal portion may have a circular internal cross-sectional profile. The first internal portion may extend from adjacent the distal end of the outer sleeve towards one or more or all arms. The first internal portion may extend past the distal ends of one or more arms.

The outer sleeve may have one or more first internal surface portions provided proximal to the distal end of the outer sleeve. The first internal surface portions, singularly or in combination, may extend around at least half of the internal perimeter of the inner surface of the outer sleeve. They may extend around at least three quarters of the internal perimeter and potentially may extend all around the internal perimeter. One or more or all of the first internal surface portions may be configured to match with external surface portions on the surgical procedural element. For instance, the first internal surface portion(s) may define a circular cross-sectional bore and the surgical procedural element may be provided with a cylindrical portion.

In use, particularly in the attached or locked state, the first internal surface portion may contact a first outside surface portion of the surgical procedural element. In use, particularly in the attached or locked state, the first internal surface portion may have a profile which corresponds to the profile of the first outside surface portion of the surgical procedural element. In use, particularly in the attached or locked state, the first internal surface portion of the outer sleeve and the first external surface portion of the surgical procedural element may cooperate to resist flexing of the mounting system and/or surgical procedural element, individually and/or relative to one another.

The outer sleeve may be mounted on a body part of the mounting system. The body part may be the inner element. The internal cross-sectional profile of the proximal end of the outer sleeve may cooperate with an external cross-sectional profile of a part of the inner element, for instance at a location intermediate the distal end of the inner element and the proximal end of the inner element.

The outer sleeve may be provided with a tapered edge at the distal end. One or more dimensions of the tapered edge, for instance the radius of the tapered edge, may decrease away from the distal end of the outer sleeve.

The outer sleeve may be provided with one or more through apertures, such as windows, extending into the gap. One or more of the through apertures may be elongate, for instance with the long axis parallel with the rotational axis of the support system. The one or more through apertures may be positioned opposite an intermediate location on the one or more arms.

The outer sleeve may have one or more internal surface portions which provide one or more surgical procedural element contacting portions.

The gap between the one or more arms and the outer sleeve may have an extent, the extent being from the distal ends of the one or more arms to the distal end face of the inner element. The extent may be at least a third of the distance between the distal face of the inner element and the distal end of the outer sleeve, potentially at least half of that distance.

The gap between the one or more arms and the outer sleeve may have a depth, the depth potentially extending radially between the one or more arms and the inner surface of the outer sleeve.

The mounting system may provide one or more torque transmission surfaces. The outer sleeve may provide one or more torque transmission surfaces. An internal surface portion of the outer sleeve may provide the one or more torque transmission surfaces. A medial facing internal surface portion may provide the one or more torque transmission surfaces. Examples of such an inward facing approach could include a polygonal or hexagonal drive. A radially extending end facing, for instance a distal end facing, internal surface portion may provide the one or more torque transmission surfaces. Examples of such an axial facing approach could include one or more face splines, for example dog clutch drives.

The outer sleeve may have one or more second internal surface portions provided proximal to the first internal surface portion. The one or more second internal surface portions may provide the one or more torque transmission surfaces. The one or more second internal surfaces may be provided proximal and/or adjacent and/or distal to the distal ends of the one or more arms.

The outer sleeve may have one or more second internal surface portions which provide one or more second surgical procedural element contacting portions. The outer sleeve, for instance by having one or more second internal surface proportions, may provide one or more surgical procedural element cooperating surfaces.

One or more inclined transition surfaces may be provided leading from one or more first internal surface portions to the one or more second internal surface portions. The distal part of the one or more inclined transition surfaces may have a lesser extent into the gap than the proximal part of the one or more inclined transition surfaces.

The portion of the outer sleeve adjacent the one or more second internal surface portions may be an annular portion of the outer sleeve. The portion of the outer sleeve adjacent the one or more second internal surface portions may have a circular external cross-section.

The one or more second internal surface portions may provide a regular cross-sectional profile. The three or more second internal surface portions may define a polygonal cross-sectional profile. The one or more second internal surface portions may extend from adjacent the distal end of the one or more arms towards the distal end of the inner element. The one or more second internal surface portions may extend from a proximal position to the distal end of the one or more arms to a more proximal position to the distal end of the one or more arms, for instance an intermediate section of the one or more arms. The one or more second internal surface portions may extend along an intermediate section of the one or more arms.

The one or more second internal surface portions, singularly or in combination, may extend around at least half of the internal perimeter of the inner surface of the outer sleeve. Potentially they may extend around at least three quarters of the internal perimeter and may extend all around the internal perimeter. One or more or all of the second internal surface portions may be configured to match with one or more second external surface portions on the surgical procedural element. For instance, six internal surface portion(s) may define a hexagonal cross-sectional bore and the surgical procedural element may be provided with a hexagonal portion, for instance adjacent the proximal end of the surgical procedural element.

The one or more second internal surface portions may provide for torque transmission from the mounting system to the surgical procedural element in use. Potentially all torque transmitted from the mounting system to the surgical procedural element is transmitted by the one or more second internal portions to the surgical procedural element, for instance to the one or more second external surface portions of the surgical procedural element.

In use, particularly in the attached or locked state, the one or more second internal surface portions may contact a corresponding second outside surface portion of the surgical procedural element. In use, particularly in the attached or locked state, the one or more second internal surface portions may have a profile which corresponds to the profile of the one or more second outside surface portion of the surgical procedural element. In use, particularly in the attached or locked state, the one or more second internal surface portions of the outer sleeve and the one or more second external surface portions of the surgical procedural element may cooperate to provide torque transmission from the mounting system to the surgical procedural element.

The one or more second internal surface portions may be provided with a varying medial extent from the inner surface of the outer sleeve at one or more different angular positions. The one or more second internal surface portions may provide a varying medial extent for the inner surface of the outer sleeve at one or more different angular positions. At least one, possibly two, potentially three, different angular positions having a first medial extent greater than a second medial extent may be provided in at least three other different angular positions. The one or more internal surface portions may have a varying medial extent, from the inner surface of the outer sleeve and/or for the inner surface, at one or more different angular positions, as the second internal surface portions:
a. have a polygonal cross-section perpendicular to the axis of rotation; or
b. have a hexagonal cross-section perpendicular to the axis of rotation; or
c. provide a spline or grooves; or
d. include two or more planar sections, such as flats, and non-planar, such as radii, surface between them; or
e. include a single abutment location, such as a key and recess type abutment.

The one or more second internal surface portions may include a face or faces in opposition to the axis of rotation. The one or more second internal surface portions may include faces in opposition to one another. The number of faces may be a match in number and/or position for cooperating faces provided by the surgical procedural element, potentially the second external surface portions thereof. The faces may have a length parallel to the axis of rotation of the mounting system. The faces may be planar and for instance with their planes each parallel to the axis of rotation.

A radially extending end facing internal surface portion may be provided with a varying axial extent. A radially extending distal end facing internal surface portion may be provided with a varying axial extent distally. The axial extent may vary at one or more different angular positions about the axis. At least one, possibly two, potentially three, different angular positions having a first axial extent greater than a second axial extent may be provided. The one or more radially extending end facing internal surface portions may have a varying axial extent as those internal surface portions:
a. have one or more face splines; or
b. provide a dog clutch; or
c. have one or more recesses, grooves or slots; or
d. have one or more protrusions, ridges or ribs.

The distal portion of one or more of the arms may include the distal end. The distal portion of one or more of the arms may include one or more surgical procedural element engagement parts. The one or more arms may have an intermediate section provided between the distal portion and/or distal ends of the one or more arms and the distal end of the inner element.

The outer sleeve may have one or more third internal surface portions provided proximal to the intermediate section of the one or more arms.

One or more second inclined transition surfaces may be provided leading from one or more second internal surface portions to the one or more third internal surface portions. The distal part of the one or more second inclined transition surfaces may have a greater extent into the gap than the proximal part of the one or more second inclined transition surfaces.

The one or more third internal surface portions, singularly or in combination, may extend around at least half of the internal perimeter of the inner surface of the outer sleeve. Potentially they extend around at least three quarters of the internal perimeter and optionally extend all around the internal perimeter. A gap may be provided between one or more or all of the third internal surface portions and the one or more arms, even with the surgical procedural element in the attached state and/or locked state.

Two or more arms that extend from the distal end of the inner element into the bore may be provided. The arms may each be spaced from the outer sleeve to define a gap between the side of the arm and the outer sleeve.

A single arm may be provided. The single arm may be parallel to the rotational axis of the mounting system. The single arm may extend from the inner element distal face. The single arm may be provided with a surgical procedural element engagement part. The engagement part may be provided at the distal end of the single arm, for instance a stem thereof. The engagement part may be provided towards the distal end of the single arm, potentially at the distal end of the single arm.

The following options may be particularly applicable to single arm embodiments. A support may be provided between the single arm and the outer sleeve. A gap, for instance to receive a part of the surgical procedural element, may be provided between the support and the outer sleeve. The gap, in use, may receive a proximal end portion of the surgical procedural element, potentially with the proximal end of the surgical procedural element being positioned between a mid-section of the single arm and a mid-section of the outer sleeve. The gap, in use, may receive a proximal end portion of the surgical procedural element, potentially with a part of the surgical procedural element being positioned between an intermediate section of the single arm and/or second internal surface portions of the outer sleeve. The locking element may provide a part of the support. The locking element may provide the support. The support may be provided without any surgical procedural element engagement parts. One or more locations of the support may face or abut the surgical procedural element, in use. A side location of the locking element may face or abut the surgical procedural element, in use. The side location may be the opposing side to the side of the locking element that faces or abuts the single arm. The support, for instance the side location, may have a point or line contact with the surgical procedural element.

One or more arms may be flexible but resilient. One or more arms may have a level of resilience to maintain their own position when unsupported. One or more arms may have a level of flexibility that their position can be altered by sliding movement of the locking element between the arm[s]. The position of the arm[s] may be altered by sliding movement of the locking element between the arm[s]. Variation in the position of the arm[s] may be resisted by the locking element when positioned between the arm[s]. The arm[s] may have a level of flexibility that their position can be altered by sliding movement of the proximal end of the surgical procedural element over the arm[s]. The position of the arm[s] may be altered by a first part of the sliding movement of the proximal end of the surgical procedural element over the arms[s] and/or the position of the arm[s] may alter during and/or after a second part of the sliding movement of the proximal end of the surgical procedural element over the arm[s]. For instance, the arm[s] may move radially inward during the first part and/or radially outward during and/or after a second part. The movement in the second part may be provided by the flexible but resilient properties of the arm[s] and/or the movement of the locking element.

The arms may be provided in opposition to one another. Potentially a single pair of arms is provided. A pair of arms may be mirror images of one another. Three arms may be provided, for instance evenly or unevenly spaced around the bore. One or more or all of the arm[s] may be parallel to the rotational axis of the mounting system.

One or more or all of the arm[s] may comprise a stem extending from the inner element distal face. One or more of all of the arm[s] may be provided with a surgical procedural element engagement part.

The engagement part may be provided at the distal end of the stem. The engagement part may be provided towards the distal end of the one or more arms, potentially at the distal end of the one or more arms.

The engagement part may include one or more abutment surfaces. The engagement part may include one or more axial movement restraining, potentially prevention, surfaces. The engagement part may include one or more limiting surfaces to limit, in use, axial movement of the mounting system and the surgical procedural element towards one another. The one or more abutment surfaces and/or axial movement restraining surfaces and/or one or more limiting surfaces may be one and the same. The one or more abutment surfaces and/or axial movement restraining surfaces and/or one or more limiting surfaces may be provided at the distal end of the one or more arms, potentially of each arm. These may provide the only limit, in use, on axial movement of the mounting system and the surgical procedural element towards one another and/or away from one another, or other such limiting surfaces may also be provided at other locations.

The engagement part may include a radially extending element. The radially extending element may have a greater radial extent than the adjacent part of the arm, such as the stem. The greater radial extent is potentially radially outward.

The engagement part may be at least partially complimentary to the profile of at least a part of a surgical procedural element engagement part. The engagement part may be complimentary to the profile of at least a part of a surgical procedural element engagement part. The radially extending element may be at least partially complimentary to the profile of at least part of a recess or groove provided by the surgical procedural element. The radially extending element may be complimentary to at least part of a recess or groove provided by the surgical procedural element.

The engagement part may include an inclined surface or face at the distal end of the engagement part, potentially with the outward radial extent increasing away from the distal end of the engagement part. The engagement part may include a second inclined surface or face at the proximal end of the engagement part, potentially with the outward radial extent decreasing away from the distal end of the engagement part.

The engagement part may be provided with an external profile that corresponds to a surgical procedural element engagement part.

Potentially the inward radial extent of the engagement part corresponds to that of the adjacent part of the arm, such as the stem. Potentially the inside surface of at least part of the arm has a consistent profile along the axis of rotation of the mounting system. The inside surface potentially defines at least a part of the second bore.

Potentially a single pair of arms are provided by the inner element, the single pair of arms axially retaining the surgical procedural element on the mounting system and the outer sleeve providing rotational drive, about the axis, from the mounting system to the surgical procedural element, in use.

The gap, in use, may receive a proximal end portion of the surgical procedural element, potentially with the proximal end of the surgical procedural element being positioned between a mid-section of the arms and a mid-section of the outer sleeve. The gap, in use, may receive a proximal end portion of the surgical procedural element, potentially with a part of the surgical procedural element being positioned between an intermediate section of the arms and/or second internal surface portions of the outer sleeve.

The gap may have a profile with the profile potentially corresponding, plus a tolerance, with the profile of the surgical procedural element at one or more locations, such as the second contact surface portions and/or second location surgical procedural element contacting portions.

The mounting system may have a mounting section at its distal end for connection to the proximal end of the surgical procedural element, in use, with the maximum dimension of the mounting section, for instance radially perpendicular to the axis of rotation, potentially being equal to or less than the maximum dimension of the surgical procedural element in a parallel plane.

The mounting section may include the section defined by the outer sleeve.

A projection of the periphery of the mounting section along the axis of rotation, may all be within the largest periphery of the surgical procedural instrument.

The maximum radial extent of the mounting section may be less than the maximum radial extent of the surgical procedural element relative to the axis of rotation.

The cross-sectional profile of the mounting section in a plane perpendicular to the axis of rotation, may be equal to or within the maximum cross-sectional profile of surgical procedural element in a parallel plane.

The mounting system may be a surgical procedural instrument mounting system. The mounting system may be a surgical instrument mounting system. The mounting system may be a surgical component mounting system.

The mounting system is potentially a rotational mounting system, with an axis of rotation extending along the long axis of the mounting system.

The mounting system may have a user interface section, potentially at the proximal end of the mounting system.

The locking element may extend from the distal end of the second bore to the user interface section.

The user interface section may include a recessed section. A second outer sleeve may be mounted in the recessed section, potentially slidably mounted. The recessed section may be an annular recess. The recessed section may extend along the axial length of the user interface section to the proximal end of the mounting system. The second outer sleeve may be an annular element. The second outer sleeve may be provided with a series of ridges and/or grooves.

The user interface section may include a slot extending into the body of the mounting system. The slot may extend to the second bore. A rib may be mounted on the second outer sleeve. The rib may extend inwardly into the slot. The second outer sleeve, potentially the rib, may engage with the locking element. For instance, the rib may be provided with a peg on the end of the rib which may engage with an aperture operably linked to the locking element.

The proximal end of the mounting system may be provided with a locking state maintenance device. The device may have a first state in which a part on the body engages with a part of the second outer sleeve, potentially to resist movement of the second outer sleeve relative to the body. The device may transition out of the first state by application of force by the user.

The locking state maintenance device may be provided on the opposite side of the body to the slot. The locking state maintenance device may be a leaf spring. The locking state maintenance device may be connected to the body at a location but spaced from the body at a free end. The free end may be provided with an outwardly extending lug that engages with an inwardly facing recess on the second outer sleeve.

The locking element may have a first state where the locking element is provided between one or more arms and/or a second state where the locking element is retracted from between one or more arms.

The locking element may have a cross-sectional profile corresponding to the cross-sectional profile of at least a part of the second bore. The locking element may be a locking shaft, potentially with a circular cross-section. The second bore may be provided with a circular cross-section, at least in part.

The locking element may be operated by the user from the proximal end of the mounting system.

In the first state, the locking element may abut the inner surface of one or more arms, potentially with the outer surface of one or more arms spaced from the inner surface of the outer sleeve. In the first state, the locking element may prevent medial direction movement of one or more arms. In the first state, the locking element may prevent movement of one or more arms and/or maintain one or more arms in a locking state.

In the second state, the locking element does not abut the inner surface of one or more arms and potentially the outer surface of one or more arms is spaced from the inner surface of the outer sleeve by the gap. In the second state, the locking element may not prevent medial direction movement of one or more arms. In the second state, the locking element may allow movement of one or more arms and/or provide one or more arms in an un-locking state.

One or more indicia may be provided on the mounting system. The one or more indicia may have an aligned state with the mounting system in an attached state and/or a misaligned state with the mounting system in a non-attached state. First indicia may be provided and the first indicia may have an aligned state when the axial position of the mounting system is in the attached state. Second indicia may be provided and the second indicia may have an aligned state with the rotational position of the mounting system is in the attached state. In use, the indicia and/or first indicia and/or second indicia may align with further indicia on the surgical procedural element when in the aligned state. In use, the indicia and/or first indicia and/or second indicia may mis-align with respect to further indicia on the surgical procedural element when in the non-aligned state.

A window may be provided in the outer sleeve through which at least a part of the gap between one or more of the arms and the inner surface of the outer sleeve can be viewed. Potentially at least one indicium is provided adjacent the window.

At least one indicium may be provided adjacent the distal end of the outer sleeve.

The first aspect of the disclosure may include any of the other features, options or possibilities set out herein, including in the other aspects of the disclosure, and including when any are taken singularly or in any combination.

According to a second aspect of the disclosure there is provided a kit, the kit comprising:
a) one or more surgical procedural elements; and
b) a mounting system for connection to one of the surgical procedural elements, the mounting system comprising;
i) an elongate body having a proximal end and a distal end;
ii) an outer sleeve being provided towards the distal end of the elongate body, the outer sleeve defining an internal bore;
iii) an inner element with a distal end facing the internal bore, the inner element providing one or more arms that extend from the distal end of the inner element into the bore, the one or more arms being spaced from the outer sleeve to define a gap between a side of one or more of the arms and the outer sleeve, the inner element at least partially defining a second bore;
iv) a locking element slidably provided in the second bore, the locking element having a first state where the locking element is provided in a first position adjacent to a distal portion of one or more of the arms and a second state where the locking element is retracted from the first position adjacent to the distal portion of one or more of the arm.

One or more or all of the surgical procedural elements may be surgical instruments. The one or more surgical procedural elements may be reamers and/or rasps and/or broaches.

The one or more surgical procedural elements may be surgical components, such as trial components and/or final components for the surgical procedure.

The proximal end of the surgical procedural element may be provided with a surgical procedural element engagement part that corresponds to the profile of an engagement part of the mounting system. The proximal end of the surgical procedural element may be provided with an internal surgical procedural element engagement part that corresponds to the profile of an internal engagement part of the mounting system, potentially the engagement part on one or more arms of the mounting system.

The surgical procedural element engagement part may be provided within a bore in the proximal end of the surgical procedural element. The bore is potentially axially aligned on the axis of rotation.

The bore may be provided with a first section having a first cross-sectional profile and a second section having a second cross-sectional profile which is large in one or more radial directions than the first. The second cross-sectional profile is potentially further from the proximal end than the first. The first cross-sectional profile may be circular. The second cross-sectional profile may be a larger circular profile.

The bore may be provided with a third cross-sectional profile which is smaller in one or more radial directions than the first and the second. The third cross-sectional profile is potentially further from the proximal end than the first and the second.

The bore may be provided with a circumferential recess or groove, potentially according to the second cross-sectional profile. The bore may be provided with an abutment surface, potentially formed by the transition to the third cross-sectional profile. The bore may be provided with a retention surface, potentially formed by the transition to the first cross-sectional profile.

The surgical procedural element engagement part may include a radially extending recess or groove. The radially extending recess or groove may have a greater radial extent than the adjacent part of the surgical procedural element engagement part. The greater radial extent is potentially radially outward.

The surgical procedural element engagement part may include an inclined surface or face at the distal end of the engagement part, potentially with the outward radial extent decreasing away from the distal end of the engagement part. The surgical procedural element engagement part may include a second inclined surface or face at the proximal end of the engagement part, potentially with the outward radial extent decreasing away from the distal end of the engagement part.

The surgical procedural element may have one or more external surface portions which provide one or more mounting system cooperating surfaces.

The one or more external surface portions, singularly or in combination, may extend around at least half of the external perimeter of the outer surface of the surgical procedural element. Potentially they extend around at least three quarters of the outer perimeter and potentially extend all around the outer perimeter. One or more or all of the external surface portions may be configured to match with second internal surface portions on the mounting system. For instance, six external surface portion(s) may define a hexagonal cross-sectional head and the mounting system may be provided with a hexagonal portion in correspondence.

The one or more external surface portions may receive torque transmission from the mounting system to the surgical procedural element in use.

The one or more mounting system cooperating surfaces may be provided with a varying radial extent from the axis of rotation at one or more different angular positions. At least three different angular positions have a first radially extent greater than the second radial extent may be provided at least three other different angular positions.

One or more indicia may be provided on a surgical procedural element. The one or more indicia may have an aligned state with the surgical procedural element in an attached state and/or a misaligned state with the surgical procedural element in a non-attached state. First indicia may be provided and the first indicia may have an aligned state when the axial position of the surgical procedural element is in the attached state. Second indicia may be provided and the second indicia may have an aligned state with the rotational position of the surgical procedural element is in the attached state. In use, the indicia and/or first indicia and/or second indicia may align with further indicia on the mounting system when in the aligned state. In use, the indicia and/or first indicia and/or second indicia may mis-align with respect to further indicia on the mounting system when in the non-aligned state. One or more of the indicia may be visible through a window provided in the outer sleeve. At least one indicium may be provided adjacent the distal end of the outer sleeve.

A portion of the proximal end of the surgical procedural element, in the attached state and/or locked state, may be provided between a portion of the outer sleeve and a portion of the one or more arms, for instance in a part of the gap.

The engagement part on one or more arms may be received in a surgical procedural element engagement part, in the attached state and/or locked state.

The locking element may prevent removal of the engagement part on one or more arms from the surgical procedural element engagement part, in the attached state and/or locked state.

The distal end of one or more arms may abut the abutment surface of the bore of the surgical procedural element in the attached state and/or locked state.

A portion of the surgical procedural element, such as the proximal end, may be visible through a window on the mounting system in the attached state and/or locked state.

The locking element may be present between one or more arms or one of the arms and the support, particularly a distal portion thereof, and/or extend into the surgical procedural element in the locked state. The locking element may be absent from between one or more arms or one of the arms and the support, particularly a distal portion thereof, and/or be retracted out of the surgical procedural element in the unlocked state and/or attached state and/or detached state.

The surgical procedural element cooperating surfaces may be engaged with the mounting system cooperating surfaces in the locked state and/or the attached state. The surgical procedural element cooperating surfaces may be disengaged from the mounting system cooperating surfaces in the detached state.

The second aspect of the disclosure may include any of the other features, options or possibilities set out herein, including in the other aspects of the disclosure, and including when any are taken singularly or in any combination.

According to a third aspect of the disclosure there is provide a method of attaching a surgical procedural element to a mounting system:
wherein the mounting system comprises: a elongate body having a proximal end and a distal end; an outer sleeve being provided towards the distal end of the elongate body, the outer sleeve defining an internal bore; an inner element with a distal end facing the internal bore, the inner element providing one or more arms that extend from the distal end of the inner element into the bore, the one or more arms being spaced from the outer sleeve to define a gap between a side of one or more of the arms and the outer sleeve, the inner element at least partially defining a second bore; a locking element slidably provided in the second bore;
the method comprising the steps of:
inserting the proximal end of the surgical procedural element into the distal end of the mounting system;
moving the locking element from a second state where the locking element is retracted from a first position adjacent to a distal portion of one or more of the arms to a first state where the locking element is provided in a first position adjacent to the distal portion of one or more of the arms.

The method may include providing, for instance inserting, a surgical procedural element engagement part into an engagement part of the mounting system. The method may include providing, for instance inserting, a surgical procedural element internal engagement part into an internal engagement part of the mounting system.

The distal ends of one or more arms of the mounting system may be provided, for instance inserted, in a bore providing the surgical procedural element engagement part.

The distal ends of one or more arms of the mounting system may be provided, for instance inserted, through a first section having a first cross-sectional profile and into a second section having a second cross-sectional profile which is large in one or more radial directions than the first.

The distal ends of one or more arms of the mounting system may be provided in, for instance brought into, abutment with a third cross-sectional profile which is smaller in one or more radial directions than the first and the second.

The engagement parts of one or more arms may be provided in, for instance brought into, a circumferential recess or groove in the surgical procedural element.

The method may include providing, for instance bringing, one or more external surface portions which provide one or more mounting system cooperating surfaces on the surgical procedural element into engagement with one or more cooperating surfaces in the mounting system.

The method may include checking the alignment of one or more indicia on the surgical procedural element with one or more indicia on the mounting system.

The method may include providing, for instance inserting, a portion of the proximal end of the surgical procedural element between a portion of the outer sleeve and a portion of one or more arms, for instance in a part of the gap.

The method may include providing, for instance inserting, the engagement part on one or more arms into a surgical procedural element engagement part.

The method may include the movement of the locking element moving, for instance deforming, one or more arms radially outward.

The method may include verifying that a portion of the surgical procedural element, such as the proximal end, is visible through a window on the mounting system in the attached state and/or locked state.

The method may include the locking element being provided, for instance inserted, between the arms, particularly a distal portion thereof, and/or being provided, for instance inserted, into the surgical procedural element in the locked state.

The method may include the surgical procedural element cooperating surfaces being engaged with the mounting system cooperating surfaces in the locked state and/or the attached state.

The third aspect of the disclosure may include any of the other features, options or possibilities set out herein, including in the other aspects of the disclosure, and including when any are taken singularly or in any combination.

According to a fourth aspect of the disclosure there is provide a method of detaching a surgical procedural element from a mounting system:
wherein the mounting system comprises: a elongate body having a proximal end and a distal end; an outer sleeve being provided towards the distal end of the elongate body, the outer sleeve defining an internal bore; an inner element with a distal end facing the internal bore, the inner element providing one or more arms that extend from the distal end of the inner element into the bore, the one or more arms being spaced from the outer sleeve to define a gap between a side of the one or more arms and the outer sleeve, the inner element at least partially defining a second bore; a locking element slidably provided in the second bore;
the method comprising the steps of:
moving the locking element from a first state where the locking element is provided in a first position adjacent to a distal portion of one or more arms to a second state where the locking element is retracted from the first position adjacent to the distal portion of one or more arms;
removing the proximal end of the surgical procedural element from the distal end of the mounting system.

The method may include removing, for instance withdrawing, a surgical procedural element engagement part from an engagement part of the mounting system. The method may include removing, for instance withdrawing, a surgical procedural element internal engagement part from an internal engagement part of the mounting system.

The distal ends of one or more arms of the mounting system may be removed, for instance withdrawn, from a bore providing the surgical procedural element engagement part.

The distal ends of one or more arms of the mounting system may be removed, for instance withdrawn, through a first section of a bore having a first cross-sectional profile and into a second section of the bore having a second cross-sectional profile which is large in one or more radial directions than the first.

The distal ends of one or more arms of the mounting system may be removed, for instance withdrawn, from abutment with a third cross-sectional profile of the bore which is smaller in one or more radial directions than the first and the second.

The engagement part of one or more arms may be removed, for instance withdrawn, from a circumferential recess or groove in the surgical procedural element.

The distal ends of one or more arms of the mounting system, particularly the engagement part[s], may be moved radially, for instance radially inward, in removing, for instance withdrawing, the surgical procedural element from the mounting system.

The method may include removing, for instance withdrawing, one or more external surface portions which provide one or more mounting system cooperating surfaces on the surgical procedural element from engagement with one or more cooperating surfaces in the mounting system.

The method may include removing, for instance withdrawing, a portion of the proximal end of the surgical procedural element from between a portion of the outer sleeve and a portion of one or more arms, for instance in a part of the gap.

The method may include removing, for instance withdrawing, the engagement part on one or more arms from a surgical procedural element engagement part.

The method may include the locking element being removed, for instance withdrawn, from between one or more arms, particularly a distal portion thereof, and/or removed, for instance withdrawn, out of the surgical procedural element in the unlocked state and/or attached state and/or detached state.

The method may include the surgical procedural element cooperating surfaces being disengaged from the mounting system cooperating surfaces in the detached state.

The fourth aspect of the disclosure may include any of the other features, options or possibilities set out herein, including in the other aspects of the disclosure, and including when any are taken singularly or in any combination.

### BRIEF DESCRIPTION OF THE DRAWINGS

Various embodiments of the disclosure will now be described, by way of example only, and with reference to the accompanying drawings in which:

FIG. 1 shows a prior art manually powered distal starter reamer;

FIG. 2a shows a prior art distal reamer and distal reamer extension therefor;

FIG. 2b shows the prior art distal reamer to distal reamer extension connection of the prior art embodiment of FIG. 2a;

FIG. 2c shows the prior art release mechanism controlling the distal reamer to distal reamer extension connection of the prior art embodiment in FIG. 2b;

FIG. 3a is a cross-sectional view through the distal end of a mounting system connected to a surgical procedural element;

FIG. 3b is a perspective cross-sectional view of the mounting system of FIG. 3a, without the surgical procedural element;

FIG. 4 shows a mounting system and a reamer connected together in the locked state;

FIG. 5a is an external view of the proximal end of a reamer adapted for use with the mounting system;

FIG. 5b is a cross-sectional view of the proximal end of FIG. 5a;

FIG. 6 shows the visual indicial used to verify correct positioning of the reamer and mounting system;

FIG. 7a is a cross-sectional view of a reamer and mounting system in the locked state;

FIG. 7b is a cross-sectional view of a reamer and mounting system in the unlocked state;

FIG. 8a is a cross-sectional view of a mounting system including the user interface section;

FIG. 8b is a perspective cross-sectional view of the user interface section of FIG. 8a;

FIG. 9a is a cross-sectional view of the mounting system without the surgical procedural element present, according to an alternative embodiment, utilising a single arm in an unlocked and detached state;

FIG. 9b is a cross-sectional view of the mounting system of FIG. 9a, with a reamer inserted into the mounting system and in the attached but still locked state; and

FIG. 9c is a cross-sectional view of the mounting system of FIGS. 9a and 9b with the locking element advanced to provide the locked state.

### DETAILED DESCRIPTION OF THE DRAWINGS

In a variety of situations, there is a need to provide a mounting system for a surgical procedural element. A particular instance where such mounting systems find use is in hip replacement procedures.
The surgical procedural element can be a surgical instrument. Reamers, broaches and rasps are all examples of such surgical instruments and hence surgical procedural elements. They find particular application in the preparation of the femur to receive a femoral component of a hip replacement. During the procedure it is necessary to prepare the femoral canal so that it is hollowed out, in the appropriate shape, to receive the stem of the femoral component.
The surgical procedural element can be a trial component. For instance, trial stems are used to check the intended position being set up for the femoral component of a hip replacement and so are examples of such surgical procedural elements.
The surgical procedural element may be a final component left in-situ after the surgical procedure, for instance the stem of the hip replacement.

Removal of the head of the femur using a saw exposes the interior of the femur. A variety of surgical instruments can be used to hollow out the femoral canal. Distal femoral canal and/or proximal femoral canal and/or intermediate femoral canal preparation may be provided. Hand operated and/or power-driven preparation may be used.

Reamers are one type of such surgical instruments. Rotation of a reamer causes blades mounted on its periphery to cut away material.

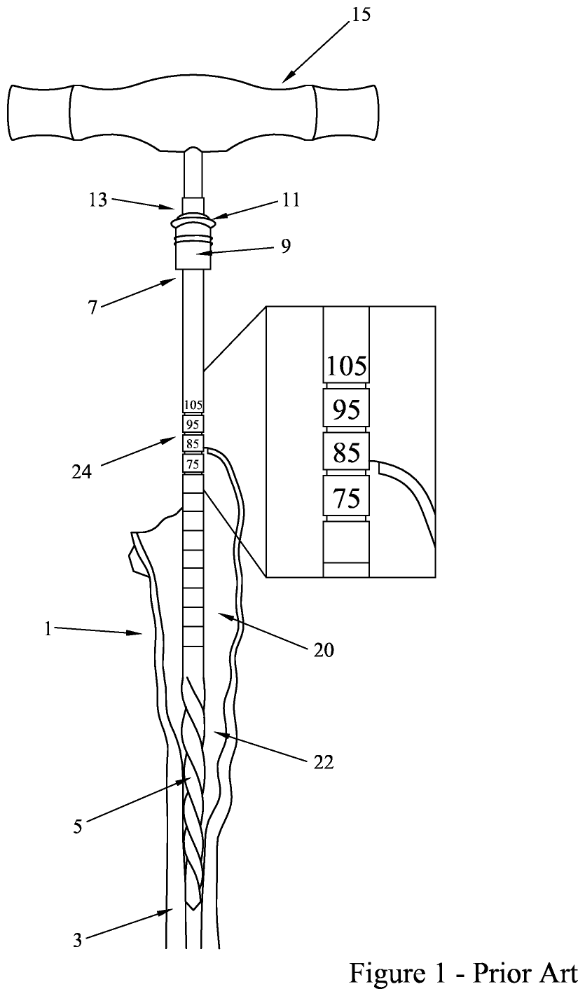

FIG. 1 is an illustration of a starter distal reamer which is used to start the process. In this case, the reamer 1 is provided with a distal end 3 where the cutting blades 5 are provided and a proximal end 7 where a first connection element 9 is provided. The first connection element 9 cooperates with a second connection element 11 provided on the distal end 13 of a manually powered force applicator 15.

In operation, the first reamer 1 is introduced to the femoral canal 20 and rotated to remove the material from the femoral canal 20. The reamer 1 is advanced axially to increase the depth of the hollow 22. The reamer 1 may be moved laterally to increase the width of the hollow 22. Marks 24 on the reamer 1 are used to allow assessment of the depth of insertion into the femoral canal 20 as reaming advances, relative to anatomical landmarks.

A set of reamers 1 is often provided with sequential increases in diameter so that the reamers 1 can be used in turn to hollow out to the desired diameter.

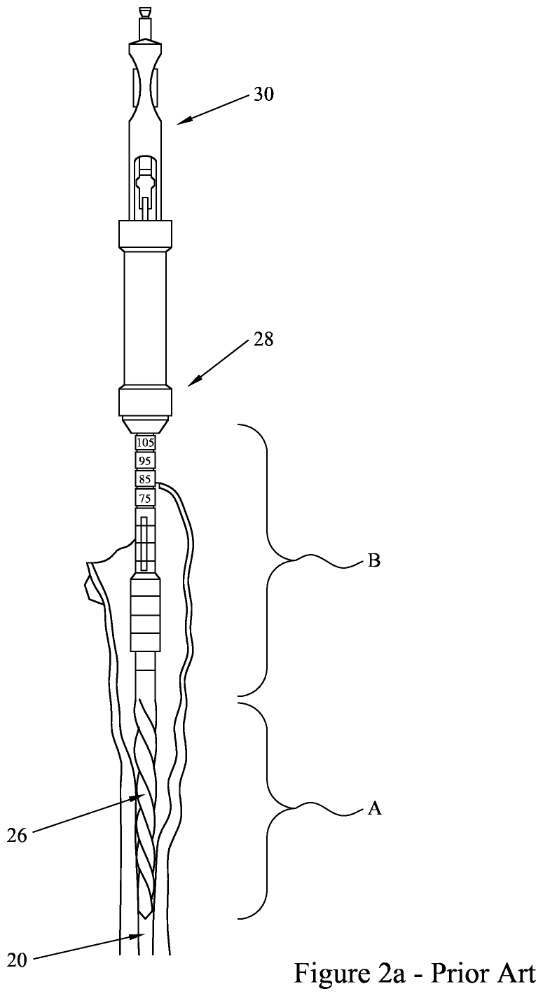

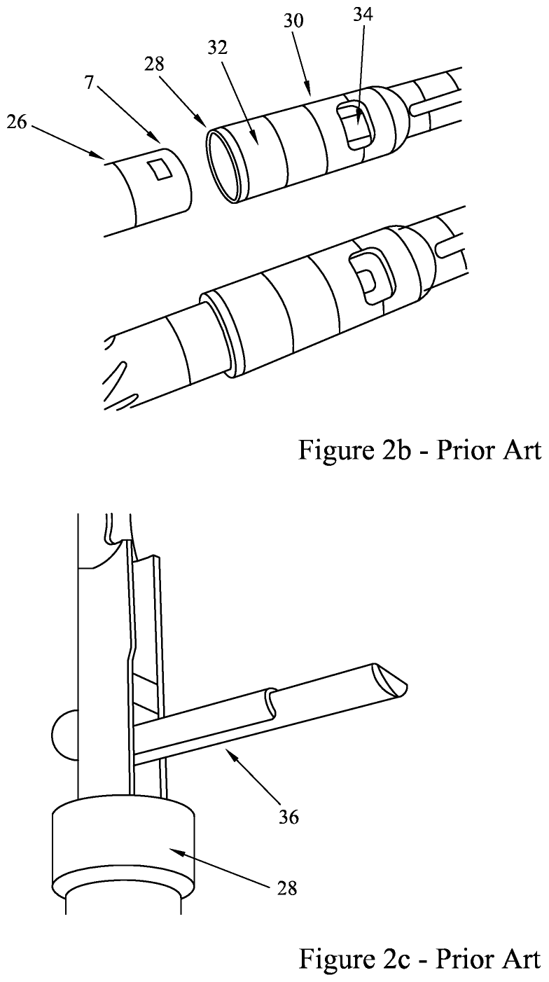

Once the use of the distal starter reamer 1 of FIG. 1 is completed, the procedure may move on to use a different distal reamer 1. Thus, the first reamer 1 is superseded by a second reamer 26, for instance the next size up and/or a different shape; see FIG. 2a.

In the example shown in FIGS. 2a and 2b, the second reamer 26 is also a distal reamer, but to enable reaming to a sufficient depth in the femoral canal 20, the second reamer 26 is mounted on the distal end 28 of an extension element 30. Again, the force applicator 15, not shown in FIG. 2a, would be connected to the proximal end, but this time of the extension element 30. The force applicator 15 may be a manually operated or may be a motive power source, such as an electric drill.

To connect the distal end 28 of the extension element 30 to the proximal end 7 of the second reamer 26, an arrangement as shown in FIG. 2b is employed. The proximal end 7 of the second reamer 26 has an external diameter less than the internal diameter of a collar 32 on the distal end 28 of the extension element 30. This allows the proximal end 7 to slide inside the extension element. A spigot and socket type arrangement is thus provided. An engagement is provided by the interaction between the L-shaped slot 34 on the proximal end 7 and an internal element [not shown].

To release the engagement, a lever 36 shown in FIG. 2c is rotated through 90º and disengages the internal element from the L-shaped slot 34. Detachment in this way may be required where a trial stem is to be left inserted to check positioning. After the trial is completed, the extension element 30 can be reconnected to the second reamer 26 to facilitate the removal of the second reamer 26 from the femoral canal 20.

A variety of different shape reamers 1, 26 can be used. Different reamer shapes are often used for the distal femoral canal preparation compared with those used in the proximal femoral canal preparation and/or intermediate femoral canal, for instance due to the different size and shape hollow 22 required for different parts of the femoral component of the hip replacement that is to be inserted.

In other situations or procedures, it is possible to use one or more broaches in the preparation of the hollow 22, one or more rasps or combinations of reamers, broaches and rasps. These can be connected to the extension element 30 in an equivalent manner.

In addition to use in forming the hollow 22, the extension element 30 and force applicator 15 can be used to introduce a trial stem and/or a final stem.

It is important that the reamer 1, 26 advances along the axis of the femoral canal 20, and hence the reamer 1, 26, extension element 30 and force applicator 15 are generally provided along a common axis to assist in this.

The reamers 1, 26 of FIGS. 1 and 2a-c all include the cutting section A and between that and the proximal end 7, a substantial section B devoid of cutting blades 5. As a result, the reamers 1,26 protrude by a material distance beyond the end of the femoral canal 20 even with the distal end 3 at its maximum insertion. This means that the connection between the distal end 28 of the extension element 30 and the proximal end 7 of the reamer 1, 26 is well outside of the femoral canal 20 and so the radial extent of the connection is not material as there is plenty of space to accommodate the connection.

Such cross-sections of connections between the extension element 30 and the surgical procedural element, exemplified by reamers 1, 26, cannot be provided at a location which will ever be within the femoral canal 20 as the cross-section, the radial extent, of the connection could exceed the hollow 22 formed and so damage or compromise the hollow 22 and hence the procedure.

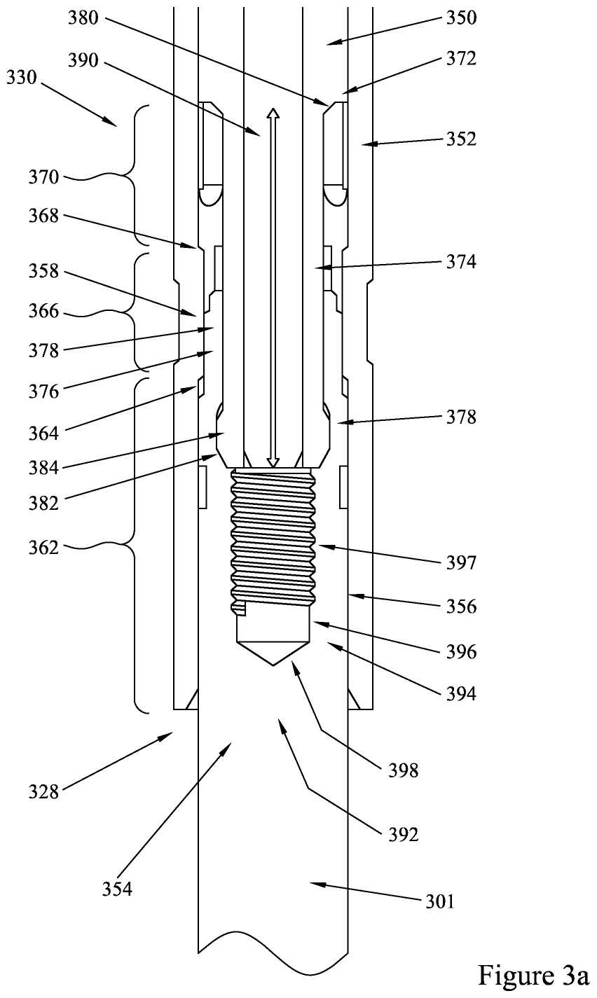

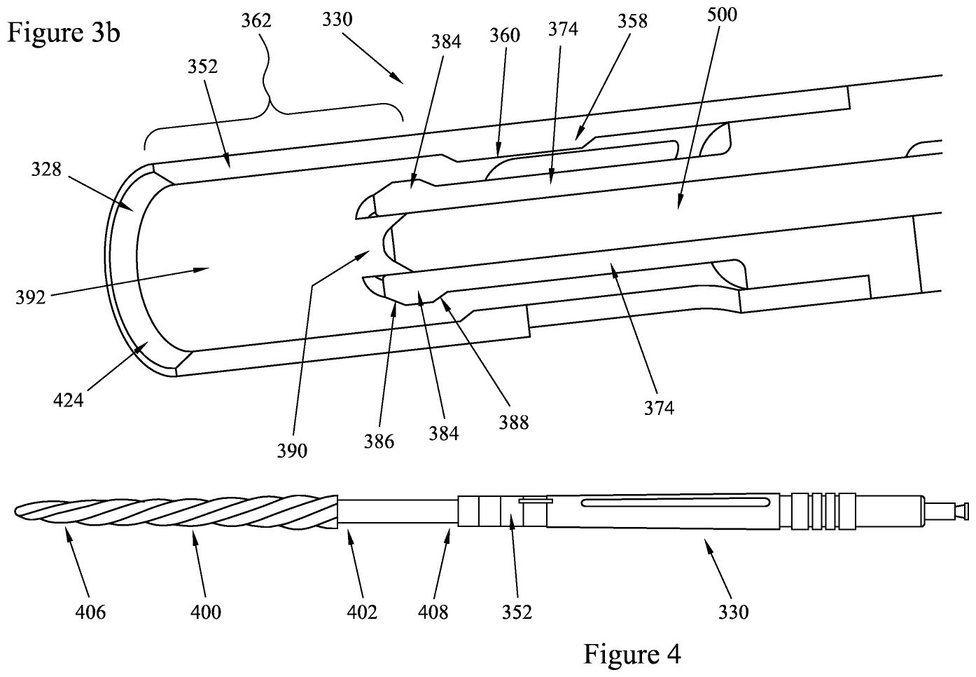

FIG. 3a shows a cross-section through the distal end 328 of a mounting system for a surgical procedural element according to the disclosure. As illustrated, the mounting system is provided at the distal end 328 of an extension element 330 to be provided between a surgical procedural element, such as a surgical instrument and a force applicator [not shown]. The surgical procedural element is to be mounted on the distal end 328 of the extension element 330.

The extension element 330 includes an annular inner sleeve 350 and an annular outer sleeve 352. The extent of the annular outer sleeve 352 defines an internal bore 354 open at the distal end 328. Away from the distal end 328, the inner surface 356 of the outer sleeve 352 has a location 358 at which a series of flat surfaces 360 are provided around the internal periphery of the outer sleeve 352. Six such flat surfaces 360 are provided, each angled at 60° to the next so as to give a hexagonal section which can interact with a location on the surgical procedural element [not shown] to apply torque thereto.

The outer sleeve internal bore 354 has a first length 362 extending inward from the distal end 328 over which the same diameter bore is provided. There is then an internal transition surface 364 leading to a second length 366 which is where the flat surfaces 360 are provided. A further internal transition surface 368 is then provided leading to a third length 370 having a bore diameter generally matching that of the first length 362 as shown, but potentially with a diameter greater than, equal to or less than the diameter of the first length 362. The length 370 provides the fitment/location to the main body.

Over the extent of the first length 362, second length 366 and third length 370, the inner sleeve 350 is absent. However, extending from the distal end 372 of the inner sleeve 350, inside the annular outer sleeve 352 and spaced therefrom, are at least two arms 374. As illustrated, a pair of arms 374 are provided that extend parallel to the longitudinal axis of the extension element 330. The outside 376 of each arm 374 is at a reduced radial distance from the longitudinal axis when compared with the inner diameter of the outer sleeve 352. Hence a gap 378 is provide. The gap 378 extends from the distal end 328 of the extension element 330 along the first length 362, second length 366 and third length 370 up to the gap end 380 where the outer diameter of the inner sleeve 350 approaches the inner diameter of the outer sleeve 352.

Towards or at the arm distal end 382 each arm 374 is provided with a radially extending section 384. This radially extending section 384 includes an insertion transition surface 386 on its distal side and a retraction transition surface 388 on its proximal side. The radially extending section 384 is provided closer to the distal end 328 of the extension element 330 than the flat surfaces 360 are in the embodiment illustrated, but they can be provided at other locations including opposite the flat surfaces 360 or more proximal than the position of the flat surfaces 360.

A central bore 390 extends along the inside of the inner sleeve 350, and hence the outer sleeve 352, and continues as the space 392 between the arms 374. As shown in FIG. 3a the space 392 is occupied by the reamer 310 provided with a proximal bore 394 which receives the arm distal ends 382. The reamer 310 occupies the gap 378 outside of the two arms 374 along this first length 362 and second length 366. The engagement is described in greater detail below.

In use, the desired surgical procedure component for use is brought into proximity with the distal end 328 of the extension element 330 and then connected.

FIG. 3b is a perspective view of the distal end 328 of the extension element 330, this time with the surgical procedural element, reamer 301, absent. The space 392 can be seen within the annular outer sleeve 352 and this has a right cylinder profile over the first length 362 before reaching the series of flat surfaces 360 that are provided around the internal periphery of the outer sleeve 352. Although the surgical procedural element is absent, the extension element is shown in the locked state present when the surgical procedural element is attached to the extension element 330 and locked in position relative to it. This locked state is provided by a locking shaft 500 which is advanced along the central bore 390 towards the distal end. When the locking shaft 500 is between the arms 374 it ensures that the radially extending sections 384 on the arms engage with and are retained in a groove on the surgical procedural element. Further details of the attached state, locked state, released state and transition between those states are set out below.

As shown in the embodiment of FIG. 4, the surgical procedural component is a reamer 400 with a body 402 on the outside of which a cutting section 404 is provided, featuring a number of cutting blades 406. The proximal end of the reamer 402 has an external profile 408 configured to be received within the inner diameter of the outer sleeve 352 of the extension element 330.

Further details of the proximal end 410 of the reamer 402 can be seen in the exterior view of FIG. 5a and cross-sectional view of FIG. 5b. Close to the proximal end 410, the reamer 402 is provided with a hexagonal cross-sectional profile 412 that is received between the series of matching flat surfaces 360 that are provided around the internal periphery of the outer sleeve 352 of the extension element 330. The initial section of the cutting blades 406 is also visible. In FIG. 5b, the profile of the axially aligned bore 414 which is configured to receive the distal ends of the arms 374 of the extension element 330 is shown. The bore 414 has an annular groove 416 profiled to match with and receive the radially extending sections 384 on the arms 374. The initial section 418 of the bore 414 has a reduced diameter compared with that of the annular groove 416. The distal end of the annular groove section 416 has a further reduced diameter section 420 which defines a stop surface 422 which resists the distal ends of the arms 374 moving too far into the bore 420, in the form illustrated, but other forms of stop or positions for the stop can be provided, such as on a shoulder at the proximal end 410.

As the extension element 330 and the reamer 400 are brought together manually by the user, the distal end 328 of the outer sleeve 352 serves to provide pre-engagement alignment of the proximal end 410 of the reamer 400 with the extension element 330. A truncated conical surface 424, seen in FIG. 3b helps with the provision of the alignment. The proximal end 410 of the reamer 400 then slides further into the space 392 at the distal end of the extension element 330, with the distal end of the arms 374 entering the bore 414 of the reamer 400. The inclined front face of the radially extending sections 384 encourages the deflection of the arms 374 together as the arms 374 enter the reduced diameter initial section 418 of the bore 414.

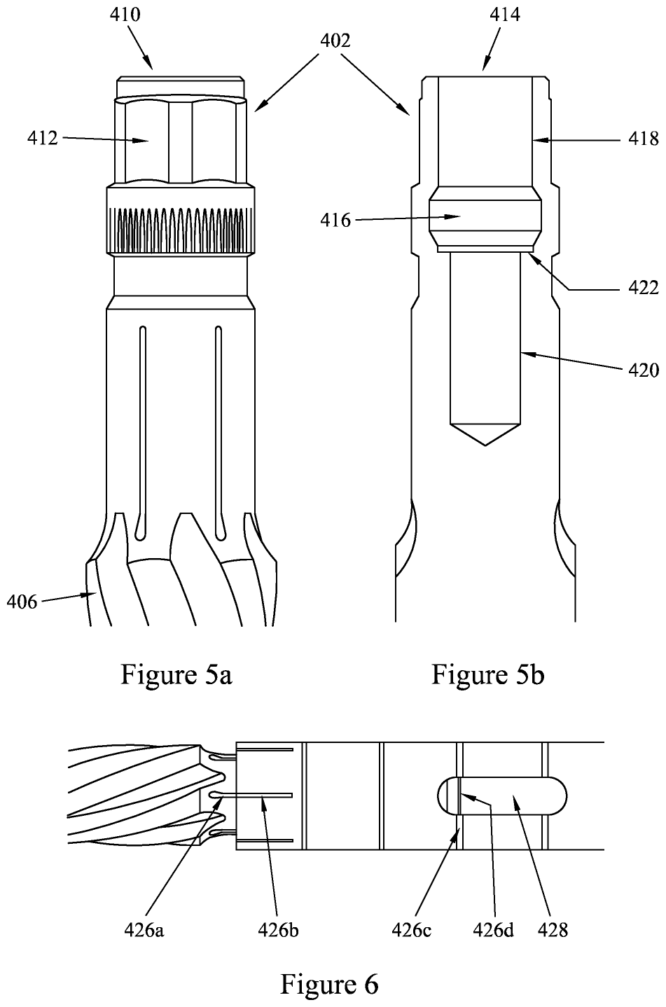

To ensure correct insertion of the reamer 400 into the extension element 330, visual indicial 426 are also provided on the external surface of the reamer 400 to provide pilot registration indicia for the reamer 400 relative to the extension element 330. As seen in FIG. 6, axially aligned indicia 426a, 426b on the two components are used to verify the correct rotary position so the hexagonal sections slot together and radially aligned indicia 426c, 426d are used, in combination with a window 428 in the outer sleeve 353, to ensure that the reamer 400 is fully inserted into the extension element 330.

In an alternative embodiment, the engraved lines acting as the visual indicia 426a, 426b and 426c, 426d, may be substituted for a flat section on each component, with the different visual appearance of the flat sections providing the visual indicia. Again, alignment of the flats to form two aligned pairs is sought in correct alignment.

The sliding advance of the reamer 400 and the extension element 330 continues with the proximal end 410 of the reamer 400 passing into the gap 378 between the arms 374 and annular outer sleeve 352. As the radially extending sections 384 reaches the annular groove 416 of the bore 414, the flexible, but resilient arms 374 are able to move outward radially and enter the annular groove 416. By this position, the proximal end 410 of the reamer 400, and more specifically the hexagonal cross-sectional profile 412 enters the second length 366 of the outer sleeve 352. As the proximal end 410 of the reamer 400 is provided with a hexagonal profile 412 that matches with the hexagonal profile defined by the flat surfaces 360 in the second length, a firm engagement is provided. The reamer 400 and extension element 330 are thus provided in the attached state. The hexagonal profile engagement resists rotation of the reamer 400 relative to the outer sleeve 352. In use, torque is applied to the reamer 400 through this interaction with the extension element 330. Before any torque is applied, however, the final transition from the attached state to the locked state is provided.

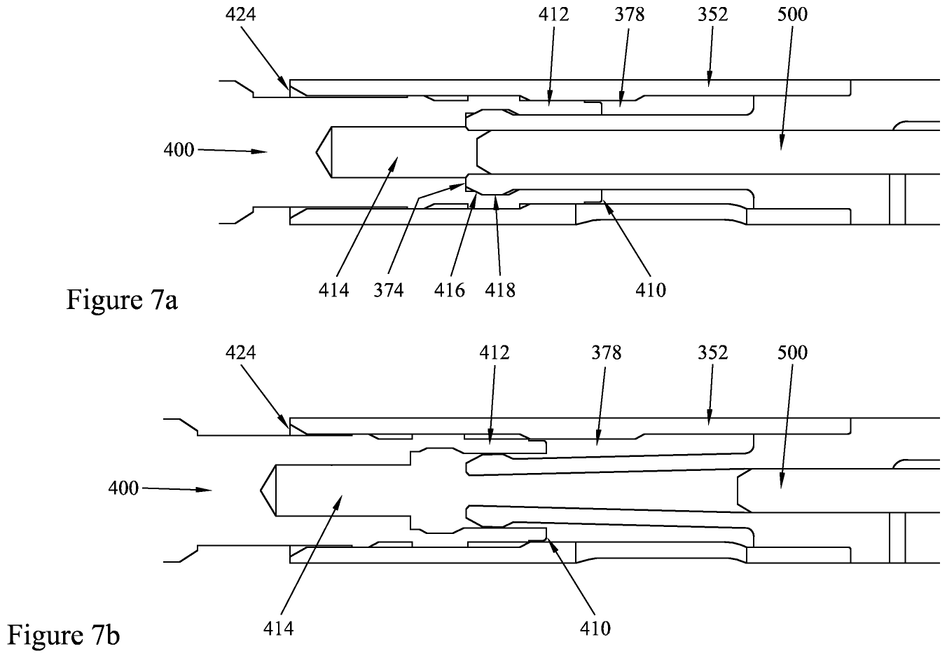

With the hexagonal profile 412 in engagement with the flat surfaces 360 and the radially extending sections 384 in engagement with the annular groove 416, the next step locks the reamer 400 on the extension element 330. Referring to FIG. 7, a locking shaft 500 is advanced down the central bore 390 into the position shown in FIG. 7. As it advances along central bore 390 no resistance to the advance is encountered. However, as the locking shaft 500 enters the space 392 between the two arms 374, the locking shaft 500 maintains the position of the arms 374 with the radially extending sections 384 in the annular groove 416, or, if necessary, encourages the full radial movement of the radially extending sections 384 outwards and into the annular groove 416. This causes a firm engagement of the radially extending sections 386 with the annular groove 416 and prevents detachment of the reamer 400 from the extension element 330; axial movement of the two, relative to each other is prevented. The locking shaft 500 and arms 374 may also assist in maintaining a firmer engagement between the hexagonal profile 412 of the reamer 400 and the hexagonal profile defined by the flat surfaces 360 of the extension element 330.

In this locked state, rotation of the extension element 330 and its outer sleeve 352 results in rotation of the reamer 400 and hence the surgical action desired. In this locked state, axial compressive force can be applied through the extension element 330 into the reamer 400 due to the firm engagement provided. Similarly, once the reamer 400 is to be removed, axial tensile force provides for withdrawal of the extension element 330 and also withdraws the firmly held reamer 400.

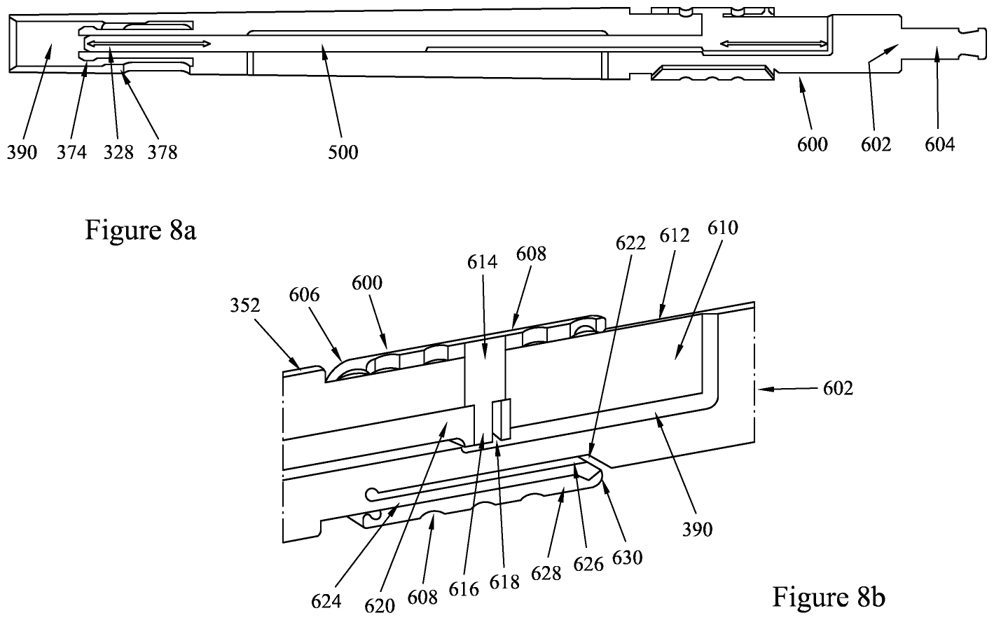

FIG. 8a provides a cross-sectional view along the length of the extension element 330. Again, the locking shaft 500 is shown in the locking state, positioned between the arms 374. The locking shaft 500 extends from the distal end 328 along to a user interface section 600 provided towards the proximal end 602 of the extension element 330. A Hudson connection 604 is provided at the proximal end 602.

The user interface section 600 is shown in more detail in FIG. 8b. A recessed section 606 is provided in the annular outer sleeve 352 in the form of an annular recess. The recessed section 606 extends the axial length of the user interface section 600 to the proximal end 602. An annular element 608 is provided in the recessed section 606 and the annular element 608 has a series of ridges and grooves to assist with grip during operation. A slot 610 extends from an entrance 612 in the recessed section 606 into the extension element 330 and reaches the central bore 390. A rib 614 extending inward from the annular element 608 has a peg 616 on the end of the rib 614 engages with an aperture 618 in the proximal end 620 of the locking shaft 500. In this way a user moving the annular element 608 away from the proximal end 602 causes the locking shaft 500 to move into the locking position between the arms 374.

On the opposite side of the recessed section 606 to the entrance 612 to the slot 610, the outer sleeve 352 provides a leaf spring 622. This is connected to the outer sleeve 352 at location 624 but spaced therefrom at the free end 626. The free end 626 is provided with an outwardly extending lug 628 that engages with an inwardly facing recess 630 when the locking shaft 500 is in the locking state. This prevents undesired axial movement of the locking shaft 500. The user can readily apply sufficient force to draw the annular element 608 back towards the proximal end 602, disengaging the recess 620 from the lug 628 and the retracting the locking shaft 500 from between the arms 374. This gives the attached state where the reamer 402 and the extension 330 are attached but only in an unlocked state. As a consequence, full compressive axial forces can be transmitted, but retention is only provided against substantially limited tensile forces and whilst full torque can be transmitted, this is not a state intended for use for torque transmission; the locked state is intended for use for torque transmission. With the locking shaft 500 retracted it is possible to apply axial force to the extension element 330 and move it away from the reamer 402 and so detach the two; a detached state.

A significant aspect of the design is apparent from FIG. 4 as the external diameter of the reamer 400 at its cutting section 406 is greater than the external diameter of the extension element 330 at the junction between the two parts and elsewhere. Hence, the insertion of the junction and other parts of the extension element 330 into the femoral canal is possible without compromising the size and shape of the hollow formed. This is in contrast to prior art systems where the junction has a materially larger profile and so must be kept out of the femur.

If it is desired to detach the extension element 330 from the reamer 400, then this can be done after removal from the patient's body or the reamer 400 can be left in-situ, for instance to facilitate trialing of the step of a femoral component of a hip replacement.

To detach, the locking shaft 500 is withdrawn into the central bore 390. With the locking shaft 500 absent, there is no compressive force encouraging the sleeve section 412 of the reamer 400 into engagement with the outer sleeve 352. Nor is the locking sleeve 500 preventing movement of the arms 374 and hence radially extending sections 384 inward towards the space 392. Thus axial separation of the reamer 400 from the extension element 330 is possible if the two are pulled in different directions.

As tensile force is applied, the hexagonal profile 412 is able to slide relative to the over the flat surfaces 360. At the same time, the retraction transition surface 388 encourages the radially extending section 384 up and out of the groove 416 and so disengages the arms 374 from the reamer 400. The reamer 400 is then free to slide out of the extension element 330.

The connection process is repeated when the reamer 400 is to be removed. Similarly, different size reamers, broaches, trial stems and the like can all be attached and detached as needed.

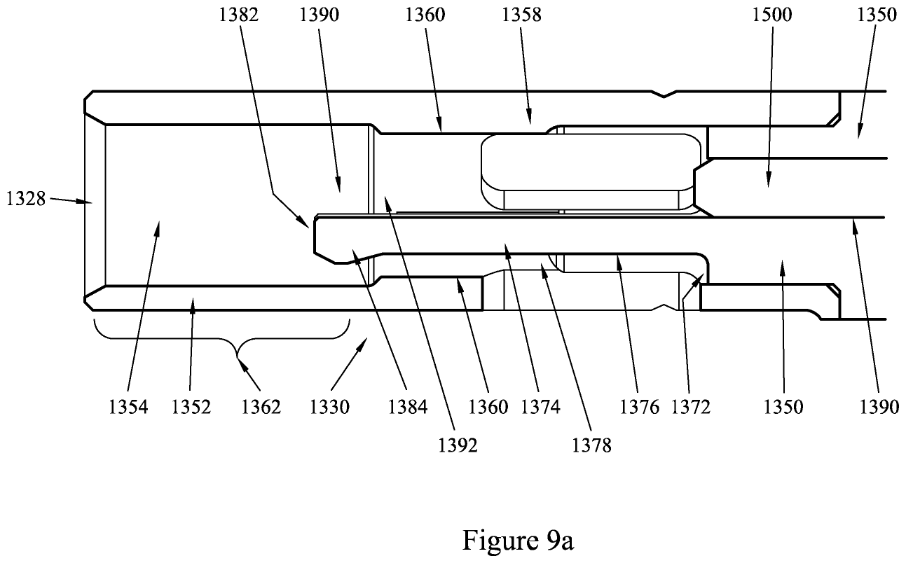

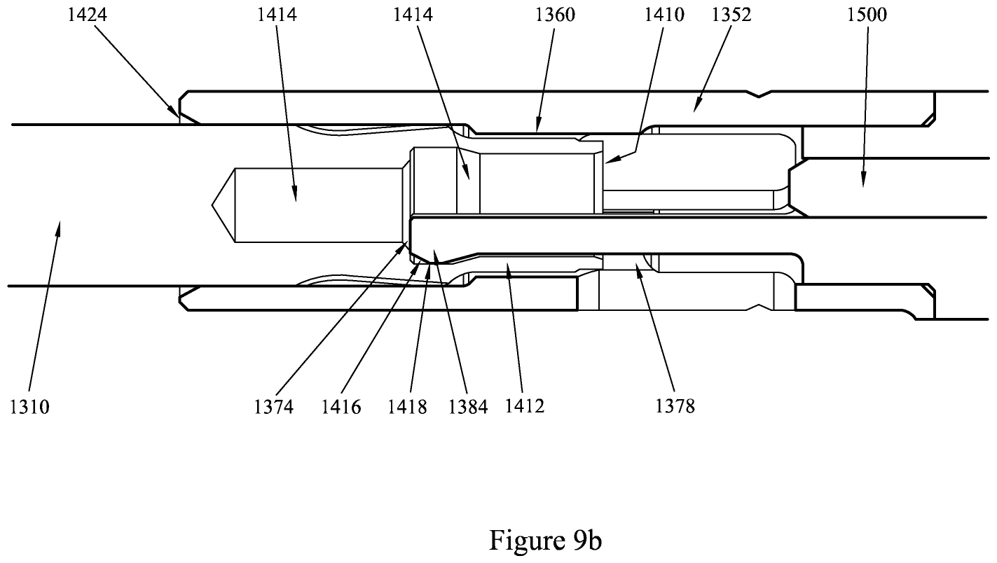

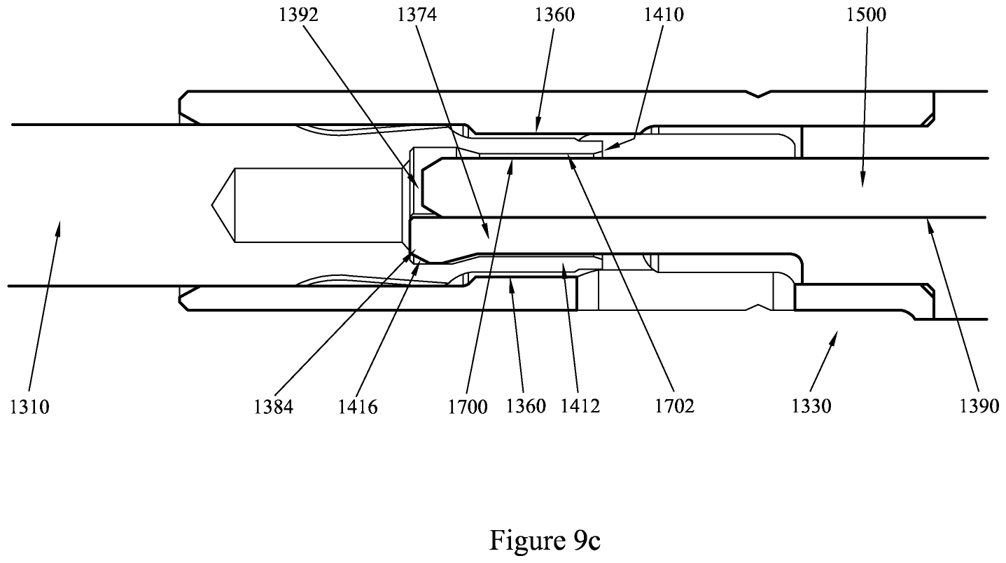

In the embodiment described above a pair of opposing arms 374 are used. It is possible to employ an asymmetric approach and FIGS. 9a and 9b illustrate one such embodiment using a single arm 900.

FIG. 9a is a perspective view of the distal end 328 of the extension element 330, with the surgical procedural element, reamer 301, absent. The space 392 can be seen within the annular outer sleeve 352 and this has a right cylinder profile over the first length 362 before reaching the series of flat surfaces 360 that are provided around the internal periphery of the outer sleeve 352. Although the surgical procedural element is absent, the extension element is shown in the locked state present when the surgical procedural element is attached to the extension element 330 and locked in position relative to it. This locked state is provided by a locking shaft 500 which is advanced along the central bore 390 towards the distal end.

In this embodiment a single arm 900 is provided. The single arm 900 is deformable, as described above, to allow for deflection medially so that the radially extending section 384 can allow the insertion of the proximal end 410 of the reamer 400, see FIG. 9b. The single arm 900 has a limited annular extent as before.

In place of a second deformable arm, this embodiment uses a collar 902 that extends from the inner sleeve 350 and has a partial annular extent. A gap 904 between the annular limit of the collar in each direction allows for relative movement of the arm 900. The collar 902 is not intended to deform and has a fixed medial position during insertion, locking, use and removal of the reamer 400. The collar may extend around 60° or more of the perimeter, potentially 180º or more. The collar 902 does not have a radially extending section 384 or other projections.

During insertion of the proximal end 410 of the reamer 400, the proximal end 410 is able to pass between the collar 902 and the inside of the outer sleeve 352 without any deformation of the collar 902. The single arm 900 is deformed medially as before.

With the hexagonal profile 412 in engagement with the flat surfaces 360 all around the perimeter of the outer sleeve 352, the reamer is ready to be locked to the extension element 330. The single radially extending section 384 on the single arm 900 is in engagement with the annular groove 416.

As shown in FIG. 9b, when the locking shaft 500 is between the arm 900 and the collar 902 it ensures that the radially extending section 384 on the arm 900 engages with and is retained in the groove 416 on the surgical procedural element. This is a more limited and asymmetric engagement than is present in the earlier embodiments but still causes a firm engagement and prevents detachment of the reamer 400 from the extension element 330; axial movement of the two relative to each other is prevented.

In this locked state, rotation of the extension element 330 and its outer sleeve 352 results in rotation of the reamer 400 and hence the surgical action desired. In this locked state, axial compressive force can be applied through the extension element 330 into the reamer 400 due to the firm engagement provided. Similarly, once the reamer 400 is to be removed, axial tensile force provides for withdrawal of the extension element 330 and also withdraws the firmly held reamer 400.

In the embodiment above, a pair of arms 374 are used in combination with a single locking shaft 500 to provide the locking state for the surgical procedural element. In this alternative embodiment, a single arm 1374 is used, as illustrated in FIGS. 9a, 9b and 9c.

FIG. 9a shows a cross-section through the distal end 1328 of a mounting system for a surgical procedural element according to the disclosure. As illustrated, the mounting system is provided at the distal end 1328 of an extension element 1330 to be provided between a surgical procedural element, such as a surgical instrument and a force applicator [not shown]. The surgical procedural element is to be mounted on the distal end 1328 of the extension element 1330.

The extension element 1330 includes an annular inner sleeve 1350 and an annular outer sleeve 1352. The extent of the annular outer sleeve 1352 defines an internal bore 1354 open at the distal end 1328. Away from the distal end 1328, the outer sleeve 1352 has a location 1358 at which a series of flat surfaces 1360 are provided around the internal periphery of the outer sleeve 1352. Six such flat surfaces 1360 are provided, each angled at 60° to the next so as to give a hexagonal section which can interact with a location on the surgical procedural element [not shown] to apply torque thereto.

The outer sleeve internal bore 1354 has a first length 1362 extending inward from the distal end 1328 over which the same diameter bore is provided.

Extending from the distal end 1372 of the inner sleeve 1350, inside the annular outer sleeve 1352 and spaced therefrom, is a single arm 1374. As illustrated, the single arm 1374 is provided such that it extends parallel to the longitudinal axis of the extension element 1330. The outside 1376 of the arm 1374 is at a reduced radial distance from the longitudinal axis when compared with the inner diameter of the outer sleeve 1352. Hence a gap 1378 is provide. The gap 1378 extends from the distal end 1328 of the extension element 1330 to where the outer diameter of the inner sleeve 1350 approaches the inner diameter of the outer sleeve 1352.

Towards or at the arm distal end 1382 of the arm 1374, it is provided with a radially extending section 1384. The radially extending section 1384 is provided closer to the distal end 1328 of the extension element 1330 than the flat surfaces 1360 are in the embodiment illustrated, but they can be provided at other locations including opposite the flat surfaces 1360 or more proximal than the position of the flat surfaces 1360.

A bore 1390 extends along the inside of the inner sleeve 1350, and hence the outer sleeve 1352, and continues in the space 1392 between the arm 374 and outer sleeve 1352. Unlike in the first embodiment above, the bore 1390 is offset relative to the central axis of the mounting system.

The sliding advance of the reamer 1310 into the extension element 1330 to reach the position of FIG. 9b continues with the proximal end 1410 of the reamer 1310 passing into the gap 1378 between the arm 1374 and annular outer sleeve 1352. As the radially extending section 1384 reaches the annular groove 1416 of the bore 1414, the flexible, but resilient arm 1374 is able to move outward radially and enter the annular groove 1416. By this position, the proximal end 1410 of the reamer 1310, and more specifically the hexagonal cross-sectional profile 1412 enters the second length of the outer sleeve 1352. As the proximal end 1410 of the reamer 1400 is provided with a hexagonal profile 1412 that matches with the hexagonal profile defined by the flat surfaces 1360 in the second length, a firm engagement is provided. The reamer 1400 and extension element 1330 are thus provided in the attached state. The hexagonal profile engagement resists rotation of the reamer 1400 relative to the outer sleeve 1352. In use, torque is applied to the reamer 1400 through this interaction with the extension element 1330. Before any torque is applied, however, the final transition from the attached state to the locked state of FIG. 9c is provided.

With the hexagonal profile 1412 in engagement with the flat surfaces 1360 and the radially extending section 1384 of the single arm 1374 in engagement with the annular groove 1416, the next step locks the reamer 1310 on the extension element 1330.

Referring to FIG. 9c, the locking shaft 1500 is advanced down the offset from centre bore 1390 into the position shown. As it advances along bore 1390 no resistance to the advance is encountered. However, as the locking shaft 1500 enters the space 1392 between the single arm 1374, the locking shaft 1500 maintains the position of the arm 1374 with the radially extending section 1384 in the annular groove 1416, or, if necessary, encourages the full radial movement of the radially extending section 1384 outwards and into the annular groove 1416.

On the side 1700 of the locking shaft 1500 away from the arm 1374, the locking shaft 1500 slides inside the bore 1414 in the proximal end 1410 of the reamer 1310. The side 1700 of the locking shaft 1500 and the offset of the locking shaft 1500 relative to the central axis of the extension element 1330 means that the side 1700 of the locking shaft 1500 is adjacent the side wall 1702 of the bore 1414.

Overall, this causes a firm engagement of the radially extending section 1386 with the annular groove 1416 and prevents detachment of the reamer 1310 from the extension element 1330; axial movement of the two relative to each other is prevented. The locking shaft 1500 and arm 1374 cooperation and the locking shaft 1500 and side wall 1702 of the bore 1414 cooperation may also assist in maintaining a firmer engagement between the hexagonal profile 1412 of the reamer 1310 and the hexagonal profile defined by the flat surfaces 1360 of the extension element 1330.

In this locked state, rotation of the extension element 1330 and its outer sleeve 1352 results in rotation of the reamer 1310 and hence the surgical action desired. In this locked state, axial compressive force can be applied through the extension element 1330 into the reamer 1310 due to the firm engagement provided. Similarly, once the reamer 1310 is to be removed, axial tensile force provides for withdrawal of the extension element 1330 and also withdraws the firmly held reamer 1310.

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

1. A mounting system for connection to a surgical procedural element, the mounting system comprising;
i) an elongate body having a proximal end and a distal end;
ii) an outer sleeve being provided towards the distal end of the elongate body, the outer sleeve defining an internal bore;
iii) an inner element with a distal end facing the internal bore, the inner element providing one or more arms that extend from the distal end of the inner element into the bore, the arm being spaced from the outer sleeve to define a gap between a side of one or more arms and the outer sleeve, the inner element at least partially defining a second bore;
iv) a locking element slidably provided in the second bore, the locking element having a first state where the locking element is provided in a first position adjacent to a distal portion of one or more of the arms and a second state where the locking element is retracted from the first position adjacent to the distal portion of the one or more arms.

2. A mounting system according to claim 1 wherein, the outer sleeve has a distal end and the one or more arm have distal ends, and wherein the distal ends of one or more of the arms is recessed within the internal bore.

3. A mounting system according to claim 1 wherein, the outer sleeve has a distal end and one or more arms have a distal end, and wherein the distal end of the outer sleeve extends beyond the distal ends of the one or more arms.

4. A mounting system according to claim 3, wherein the distal end of the outer sleeve extends beyond the distal ends of the one or more arms by at least a quarter of the length of one or more of the arms.

5. A mounting system according to claim 1, wherein the outer sleeve has one or more internal surface portions, proximal to the distal end of the outer sleeve, which provide one or more surgical procedural element contacting portions.

6. A mounting system according to claim 5, wherein the one or more surgical procedural contacting portions, in combination, extend around at least half of the internal perimeter of the inner surface of the outer sleeve.

7. A mounting system according to claim 1, wherein the gap between one or more arms and the outer sleeve has an extent, the extent being from the distal ends of the one or more arms to the distal end face of the inner element, and wherein the extent is at least a third of the distance between the distal face of the inner element and the distal end of the outer sleeve.

8. A mounting system according to claim 1, wherein the outer sleeve has one or more second location internal surface portions which provide one or more second location surgical procedural element contacting portions.

9. A mounting system according to claim 8, wherein the one or more second location surgical procedural contacting portions, in combination, extend around at least half of the internal perimeter of the inner surface of the outer sleeve.

10. A mounting system according to claim 1, wherein the one or more arms have an intermediate section between the distal ends of the one or more arms and the distal end of the inner element, and wherein the outer sleeve has one or more third internal surface portions, proximal to the intermediate section of the one or more arms.

11. A mounting system according to claim 1 wherein, the gap includes a gap profile that corresponds, plus a tolerance, with the profile of the surgical procedural element at one or more locations, such as the second location surgical procedural element contacting portions and/or third location surgical procedural element contacting portions.

12. A mounting system according to claim 1, wherein in the first state, the locking element abuts the inner surface of one or more arms and the outer surface of one or more arms is spaced from the inner surface of the outer sleeve.

13. A mounting system according to claim 1, wherein in the second state, the locking element does not abut the inner surface of the one or more arms and the outer surface of the one or more arms is spaced from the inner surface of the outer sleeve by the gap.

14. A mounting system according to claim 1, wherein a single pair of arms are provided by the inner element, the single pair of arms axially retaining the surgical procedural element on the mounting system and the outer sleeve providing rotational drive, about the axis, from the mounting system to the surgical procedural element, in use.

15. A mounting system according to claim 1, wherein surgical procedural element cooperating surfaces are provided by the outer sleeve.

16. A mounting system according to claim 15, wherein the cooperating surfaces have a varying medial extent from the inner surface of the outer sleeve at one or more different angular positions.

17. A mounting system according to claim 16, wherein at least three different angular positions have a first medial extent greater than the second medial extent provided at least three other different angular positions.

18. A mounting system according to claim 16, wherein the cooperating surfaces have a varying medial extent from the inner surface of the outer sleeve at one or more different angular positions, as the cooperating surfaces:
a. have a polygonal cross-section perpendicular to the axis of rotation; or
b. have a hexagonal cross-section perpendicular to the axis of rotation; or
c. provide a spline; or
d. include two or more planar sections, such as flats, and non-planar, such as radii, surface between them; or
e. include a single abutment location, such as a key and recess type abutment.

19. A mounting system according to claim 17, wherein the cooperating surfaces have a length parallel to the axis of rotation.

20. A mounting system according to claim 17, wherein the cooperating surface are planar and the planes are parallel to the axis of rotation.

21. A mounting system according to claim 15, wherein the cooperating surfaces have a varying axial extent at one or more different positions.

22. A mounting system according to claim 21, wherein at least two different angular positions have a first axial extent greater than a second axial extent.

23. A mounting system according to claim 21, wherein the cooperating surfaces have a varying axial extent at one or more different positions, as the cooperating surface:
a. have one or more face splines; or
b. provide a dog clutch; or
c. have one or more recesses, grooves or slots; or
d. have one or more protrusions, ridges or ribs.

24. A mounting system according to claim 22, wherein the mounting system has a mounting section at its distal end for connection to the surgical procedural element in use, the maximum dimension of the mounting section, radially perpendicular to the axis of rotation, being equal to or less than the maximum dimension of the surgical procedural element in a parallel plane.

25. A mounting system according to claim 22, wherein the cross-sectional profile of the mounting section in a plane perpendicular to the axis of rotation, is equal to or within the maximum cross-sectional profile of surgical procedural element in a parallel plane.

26. A mounting system according to claim 1, wherein indicia are provided on the mounting system and wherein the indicia have an aligned state with the mounting system in an attached state and a misaligned state with the mounting system in a non-attached state.

27. A mounting system according to claim 26, wherein first indicia are provided and the first indicia have an aligned state when the axial position of the mounting system is in the attached state and/or wherein second indicia are provided and the second indicia have an aligned state with the rotational position of the mounting system is in the attached state.

28. A mounting system according to claim 26, wherein, in use, the indicia and/or first indicia and/or second indicia align with further indicia on the surgical procedural element in the aligned state.

29. A mounting system according to claim 26, wherein a window is provided in the outer sleeve through which, in the attached state, at least a part of the surgical procedural element can be viewed, at least one indicia being provided adjacent the window.

30. A mounting system according to claim 26, wherein at least one indicium is provided adjacent the distal end of the outer sleeve.

31. A surgical kit, the surgical kit comprising:
a) one or more surgical procedural elements; and
b) a mounting system for connection to one of the surgical procedural elements, the mounting system comprising;
i) an elongate body having a proximal end and a distal end;
ii) an outer sleeve being provided towards the distal end of the elongate body, the outer sleeve defining an internal bore;
iii) an inner element with a distal end facing the internal bore, the inner element providing one or more arms that extend from the distal end of the inner element into the bore, the one or more arms being spaced from the outer sleeve to define a gap between a side of one or more of the arms and the outer sleeve, the inner element at least partially defining a second bore;
iv) a locking element slidably provided in the second bore, the locking element having a first state where the locking element is provided in a first medial position adjacent to a distal portion of one or more of the arms and a second state where the locking element is retracted from the first position adjacent relative to the distal portion of one or more of the arms.

32. The kit of claim 29, wherein one or more or all of the surgical procedural elements are surgical instruments, the one or more surgical procedural elements including reamers and/or rasps and/or broaches.

33. The kit of claim 29, wherein the proximal end of the surgical procedural element is provided with a surgical procedural element engagement part that corresponds to the profile of an engagement part of the mounting system.

34. The kit of claim 29, wherein the surgical procedural element engagement part is provided within a bore in the proximal end of the surgical procedural element, the bore being provided with a first section having a first cross-sectional profile and a second section, distal to the first section, having a second cross-sectional profile which is large in one or more radial directions than the first.

35. The kit of claim 29, wherein the surgical procedural element has one or more external surface portions which provide one or more mounting system cooperating surfaces, the one or more external surface portions receiving torque transmission from the mounting system to the surgical procedural element, in use.

36. The kit of claim 29, wherein:
a. a portion of the proximal end of the surgical procedural element, in the attached state and/or locked state, is provided between a portion of the outer sleeve and a portion of the arms; and/or
b. the engagement part on the arms is received in a surgical procedural element engagement part, in the attached state and/or locked state; and/or
c. the locking element prevents removal of the engagement part on the arms from the surgical procedural element engagement part, in the locked state.

37. A method of attaching a surgical procedural element to a mounting system:
wherein the mounting system comprises: a elongate body having a proximal end and a distal end; an outer sleeve being provided towards the distal end of the elongate body, the outer sleeve defining an internal bore; an inner element with a distal end facing the internal bore, the inner element providing one or more arms that extend from the distal end of the inner element into the bore, the one or more arms being spaced from the outer sleeve to define a gap between a side of one or more of the arm and the outer sleeve, the inner element at least partially defining a second bore; a locking element slidably provided in the second bore;
the method comprising the steps of:
inserting the proximal end of the surgical procedural element into the distal end of the mounting system;
moving the locking element from a second state where the locking element is retracted from a first medial position adjacent to a distal portion of one of more of the arms to a first state where the locking element is provided in the first position adjacent to distal portion of one or more of the arm.

38. A method of detaching a surgical procedural element to a mounting system:
wherein the mounting system comprises: a elongate body having a proximal end and a distal end; an outer sleeve being provided towards the distal end of the elongate body, the outer sleeve defining an internal bore; an inner element with a distal end facing the internal bore, the inner element providing one or more arms that extend from the distal end of the inner element into the bore, the one or more arms being spaced from the outer sleeve to define a gap between a side of the one or more arms and the outer sleeve, the inner element at least partially defining a second bore; a locking element slidably provided in the second bore;
the method comprising the steps of:
moving the locking element from a first state where the locking element is provided in a first position adjacent to a distal portion of one or more of the arms to a second state where the locking element is retracted from the first position adjacent to the distal portion of the one or more of the arm;
removing the proximal end of the surgical procedural element from the distal end of the mounting system.
